(19) United States
(12) Patent Application Publication
(10) Pub. No.: US 2008/0262433 A1
(43) Pub. Date: Oct. 23, 2008

(54) INTRATHECAL INJECTION GUIDE APPARATUS AND METHODS FOR MAKING AND USING SAME

(75) Inventors: Jin Mo Chung, Galveston, TX (US); Inhyung Lee, Seoul (KR); Hee Young Kim, Galveston, TX (US)

Correspondence Address: ROBERT W STROZIER, P.L.L.C, PO BOX 429, BELLAIRE, TX, 77402-0429, US

(73) Assignee: The Board of Regents of The University of Texas System, Austin, TX (US)

(21) Appl. No.: 12/105,422

(22) Filed: Apr. 18, 2008

Related U.S. Application Data
(60) Provisional application No. 60/912,556, filed on Apr. 18, 2007.

(51) Int. Cl. A61M 5/32

(52) U.S. Cl. 604/174; 604/272

(57) ABSTRACT

An intrathecal injection apparatus is disclosed for use with small animals. The apparatus includes an animal conforming member and a guide tube for inserting a needle and directing the needle so that it properly penetrates the spinal sack of the animal.

### RELATED APPLICATIONS

This application claims priority to and the benefit of U.S. Provisional Patent Application Ser. No. 60/912,556, filed Apr. 18, 2007, incorporated herein by reference.

### GOVERNMENTAL INTEREST

Governmental entities may have certain rights in and to the contents of this application due to funded from the National Institute of Health grants AT001474 and NS31680.

### BACKGROUND OF THE INVENTION

1. Field of the Invention

The present invention relates to an intrathecal injection guide apparatus for use with small animals to assist in transferring fluids in and out of the animals spinal fluid and for methods for making and using same.

More particularly, the present invention relates to an intrathecal injection guide apparatus for use with small animals to assist in transferring fluids in and out of the animal's spinal fluid, where the apparatus includes a member adapted to conform to a portion of a lower torso of the animal fitted with two opposing member adapted to engage opposing anatomical features on of the animal's lower torso. The apparatus also includes a needle guide including a needle guide tube therethrough disposed on a top surface of the animal conforming member adapted to have a needle inserted into the tube in a proper trajectory or alignment to penetrate into the spinal fluid sack at its distal end, which lacks major neuron bundles. The guide may include a needle housing having a needle pre-positioned in the housing with its shaft end disposed in the tube, where the needle can be attached to an empty or filled reservoir prior to the needle tip be directed into the spinal sack. The invention also relates to methods for making and using same.

2. Description of the Related Art

Performing intrathecal injection in to small animals such as mice and rats is very challenging. Often times, it may take several attempts to properly insert and position a needle. Empirically, only about 50% of insertions are initially successful. No such tool is available from our own long experience with animal research with rats and mice. Since intrathecal injection is a common requirement in animal research and is a difficult procedure to learn, this device would provide a means to successfully complete a difficult task in a minimum amount of time, thereby providing the researcher more time to focus on other tasks.

Thus, there is a need in the art for an apparatus to improve intrathecal injections into small animals and a method for using the apparatus.

### SUMMARY OF THE INVENTION

The present invention provides an intrathecal injection guide apparatus for use with small animals to assist in transferring fluids in and out of the animal's spinal fluid, where the apparatus includes a member adapted to conform to a portion of a lower torso of the animal fitted with two opposing member adapted to engage opposing anatomical features on of the animal's lower torso to properly position the conforming member to the animal. The apparatus also includes a needle guide including a needle guide tube passing through the guide and the member and disposed on a top surface of the conforming member. The guide is adapted to have a needle inserted into the tube, where the tube defines a proper trajectory or alignment. When the apparatus is fitted onto an animal, the needle is simply pushed to penetrate into the spinal fluid sack at the sack's distal end, which lacks major neuron bundles.

The present invention also provides an intrathecal injection guide apparatus for use with small animals to assist in transferring fluids in and out of the animal's spinal fluid, where the apparatus includes a member adapted to conform to a portion of a lower torso of the animal fitted with two opposing member adapted to engage opposing anatomical features on of the animal's lower torso to properly position the conforming member to the animal. The apparatus also includes a needle guide including a needle guide tube passing through the guide and the member and disposed on a top surface of the conforming member. The guide also includes a needle housing having a needle pre-positioned in the housing with its shaft end disposed in the tube, where the needle can be attached to an empty or filled reservoir prior to a needle tip being directed into the spinal sack. The guide is adapted to have a needle inserted into the tube, where the tube defines a proper trajectory or alignment. When the apparatus is fitted onto an animal, the needle is simply pushed to penetrate into the spinal fluid sack at the sack's distal end, which lacks major neuron bundles.

The present invention also provides a method of transferring fluids in and out of the animal's spinal fluid including the step of placing the conforming member of the intrathecal injection guide apparatus of this invention on a small animal so that two opposing member engage the two opposing anatomical features of the animal. Once properly place on the animal, a needle is inserted into the guide tube of the guide and forced into the spinal sack of the animal in a distal location of the sack. After the needle is inserted, the proximal end of the needle is attached to a empty of filled reservoir. After attachment, material can either be injected into or removed from the sack.

### DESCRIPTION OF THE DRAWINGS

The invention can be better understood with reference to the following detailed description together with the appended illustrative drawings in which like elements are numbered the same:

FIG. 1A depicts a front view of an embodiment of an intrathecal injection apparatus of this invention.

FIG. 1B depicts a photograph of a front view of the apparatus of FIG. 1A.

FIG. 1C depicts a side view of the apparatus of FIG. 1A.

FIG. 1D depicts a photograph of a side view of the apparatus of FIG. 1A.

FIG. 1E depicts a rear view of the apparatus of FIG. 1A.

FIG. 1F depicts a photograph of an oblique view of the apparatus of FIG. 1A.

FIG. 2 depicts a front view of another embodiment of an intrathecal injection apparatus of this invention.

FIG. 3 depicts a front view of another embodiment of an intrathecal injection apparatus of this invention.

### DETAILED DESCRIPTION OF THE INVENTION

The inventors have found that an apparatus can be constructed that helps provide error-free intrathecal injections in rats, mice or other small animals. The apparatus includes an animal conforming plate or member having a needle guide including a needle guide tube. The apparatus is adapted to fit on a dorsal surface of the animal so that a needle can be inserted through the tube and directly into the spinal sack for injecting or withdrawing fluids from the spinal cord sack.

The invention broadly relates to an intrathecal injection apparatus including an animal conforming member adapted to conform to a back portion of an animals body like a saddle. The apparatus also include a needle guide tube. The apparatus also includes two opposing apertures adapted to engage rear hip joints of the animal or other readily identifiable opposing anatomical feature so that the guide tube is properly aligned with distal portion of the animal spinal sack. The apparatus can also include a tube mount. The apparatus can also include a needle housing affixed to the conforming member and adapted to house a needle so that when properly positioned a tip of the needle can be inserted into the spinal sack.

This invention is an apparatus that is used to guide intrathecal injection in small animals such as rodents. A fitted plastic plate or member is designed to cover the dorsal surface of a rodent, attached to each side of the plate is a ring that serves as a guide to anchor both the back and hip bones. At a specific position on the plate, there is a small tube where a hyperdermic needle will be fitted at a specified angle. The device is designed to provide for successful intrathecal injection of a compound by a relatively untrained animal researcher using several simple steps: 1) place the plate on the dorsal surface making sure the back and hip bones fit into the correct position on the molded plate, 2) insert a hyperdermic needle filled with a compound into the needle tube, 3) advance the needle for a specified distance, and 4) inject the compound. A set of several different sizes of devices are to be designed for use on several different sizes of rats and mice, based on their weight.

In one embodiment, the present invention relates to an intrathecal injection guide apparatus including a body member adapted to conform to a portion of a lower torso of a small animal fitted with two opposing member adapted to engage opposing anatomical features on of the animal's lower torso to properly position the conforming member to the animal. The apparatus also include a guide member disposed on a top surface of the body member, and a needle guide tube passing through the guide member and the body member. The tube defines a proper needle trajectory or alignment so that when the apparatus is fitted onto the animal, a needle can simply be pushed to penetrate a spinal fluid sack of the animal at its distal end, where the sack lacks major neuron bundles. In certain embodiments, the needle guide and the conforming member are a unitary assembly. In other embodiments, the needle guide is fixedly attached to the top surface of the conforming member. The apparatus can also include an empty or filled reservoir attached to a proximal end of the needle and adapted to permit a spinal material to be removed from the sack or a treating material to be injected into the sack.

In another embodiment, the present invention relates to an intrathecal injection guide apparatus including a body member adapted to conform to a portion of a lower torso of a small animal fitted with two opposing member adapted to engage opposing anatomical features on of the animal's lower torso to properly position the conforming member to the animal. The apparatus also includes a guide member disposed on a top surface of the body member, and a tube passing through the guide member and the body member. The guide member includes a needle housing having a needle pre-positioned in the housing with its tip positioned in a distal end of the tube. The tube defines a proper needle trajectory or alignment so that when the apparatus is fitted onto the animal, the needle can simply be pushed to penetrate a spinal fluid sack of the animal at its distal end, where the sack lacks major neuron bundles. In certain embodiments, the needle guide and the conforming member are a unitary assembly. In other embodiments, the needle guide is fixedly attached to the top surface of the conforming member. The apparatus can also include an empty or filled reservoir attached to a proximal end of the needle and adapted to permit a spinal material to be removed from the sack or a treating material to be injected into the sack.

In one embodiment, the present invention relates to a method of transferring fluids in and out of the animal's spinal fluid including the step of fitting on a small animal an apparatus of this invention and inserting a needle into the guide tube of the guide member. The method can also include the step of forcing the needle into a spinal sack of the animal in a distal location of the sack. In certain embodiments, the method also includes the steps of attaching a proximal end of the needle to an empty or filled reservoir, and either injecting a treating material into the sack or removing a spinal material from the sack. In another embodiment, the method also includes the steps of attaching a proximal end of the needle to an empty reservoir, and removing a spinal material from the sack. In another embodiment, the method also includes the steps of attaching a proximal end of the needle to a filled reservoir, and injecting a treating material into the sack.

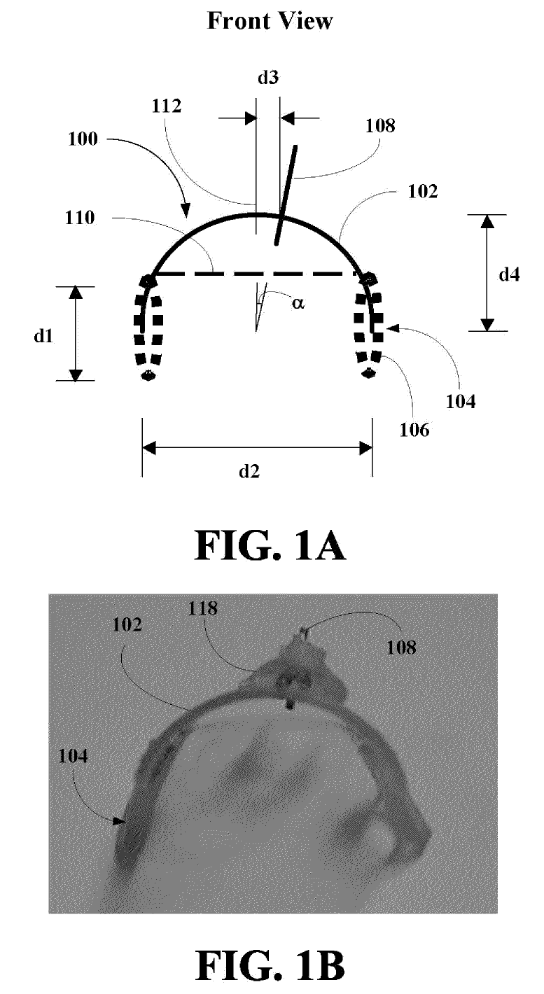

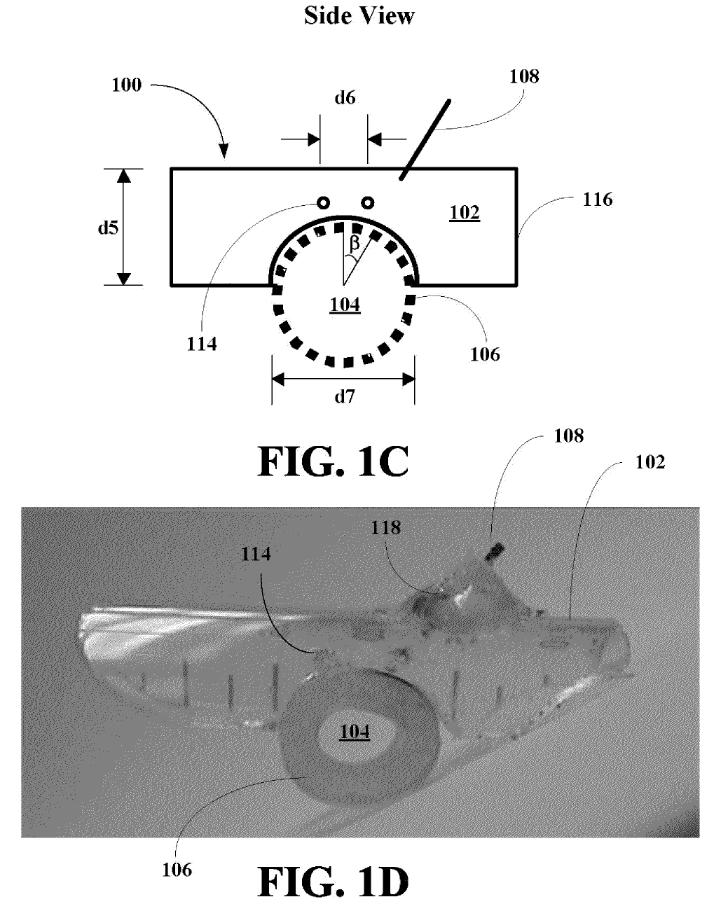

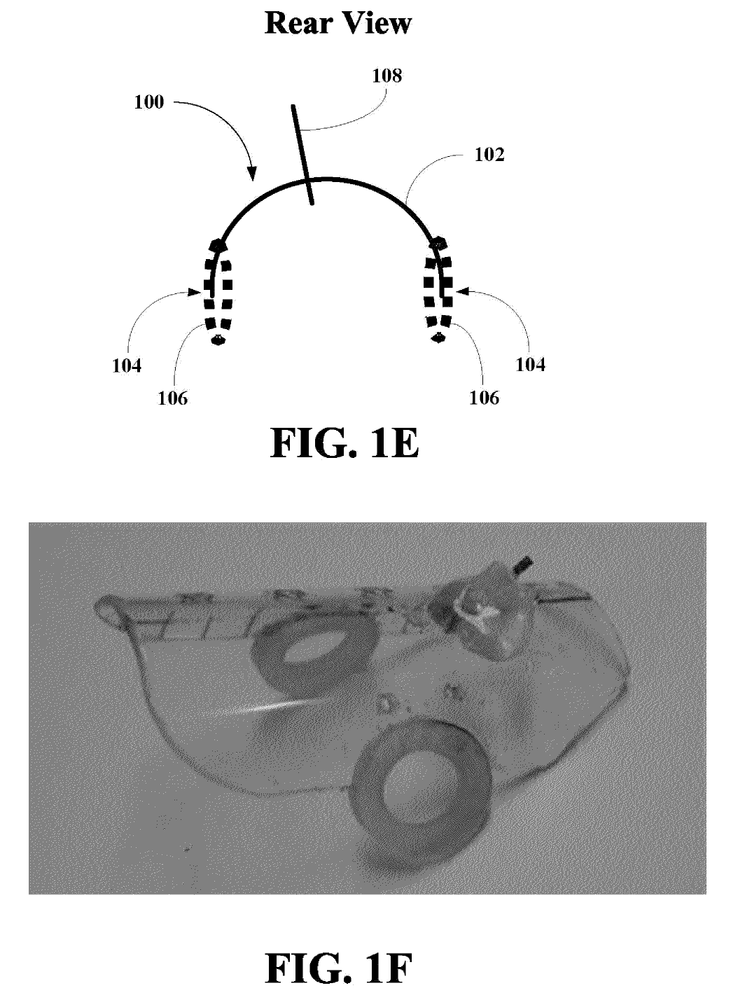

Referring now to FIGS. 1A&B, an embodiment of an intrathecal injection apparatus, generally 100, is shown to include a conforming member 102 adapted to conform to a portion of an animal back. The apparatus 100 also include two opposing apertures 104 surrounded by a flexible material member 106, where the two apertures 104 are shown here extending downward from the member 102. The apparatus 100 also include a needle guide tube 108. The apparatus 100 also include a fastening member or string 110. The apparatus 100 also includes a tube mount 118 adapted to mount the tube 108 to the member 102.

The tube 108 is angled from the vertical by an angle α. The angle α is adapted to ensure that a needle inserted into the tube 108 will be guide properly in a distal portion of the spinal sack of the animal. Generally, the angle α is between about 5° and about 15°. The tube 108 is also offset from a center 112 of the member 102 by an amount d3. The distance d3 is also adapted to ensure that a needle inserted into the tube 108 will be guide properly in a distal portion of the spinal sack of the animal. Generally, the distance d3 is between about 2 mm to about 8 mm. The conforming member 102 is adapted to conform to a given small animal. In the case of any small animal, the conforming member 102 is defined by three size parameters d1, d2 and d4. The size parameter d1 represents a diameters of the conforming member 102 or their surrounding members 106. The size parameter d2 represents a width of the conforming member 102. The size parameter d4 represent a height of the conforming member 102. d1 and d7 are equal and range from about 4 mm to about 15 mm. For mice, d2 ranges from about 5 mm to about 20 mm and for rats, d2 ranges from about 20 mm to about 40 mm. d4 and d5 are equal; for mice, d4 ranges from about 3 mm to about 10 mm and for rats, d4 ranges from about 10 mm and about 20 mm.

Referring now to FIGS. 1C&D, a side view of the apparatus 100 is shown. The apparatus 100 also includes four string apertures 114, two on one side of the member 102 and two on the other side of the member 102. The four spring apertures 114 are adapted to receive a fastening member or string 110. The fastening member 110 is designed to hold the apparatus 100 in place on the animal along with the two members 106. The design characteristics of the apparatus 100 are also defined by two more size parameter d5 and d6. The parameter d5 is the same or different as the parameter d4 and have the same general ranges. The parameter d6 represents a distance between the pair of apertures 114, and ranges from about 3 mm to about 9 mm. As shown in FIG. 1B, the tube 108 is also angled with respect to the center 112 toward a rear 116 of the member 102 by an angle β. The angle β like the angle α is a design parameters that is adapted to properly align the tube 108 so that needle inserted into the tube can penetrate into the spinal sack of the animal near its distal end, furthest portion of the spinal sack from the brain stem. β (beta) ranges from about 40° to about 50°. The apparatus 100 also includes a tube mount 118 adapted to mount the tube 108 to the member 102.

Referring now to FIG. 1E, a rear view of the apparatus 100 is shown, where FIG. 1F is an oblique view of the apparatus 100.

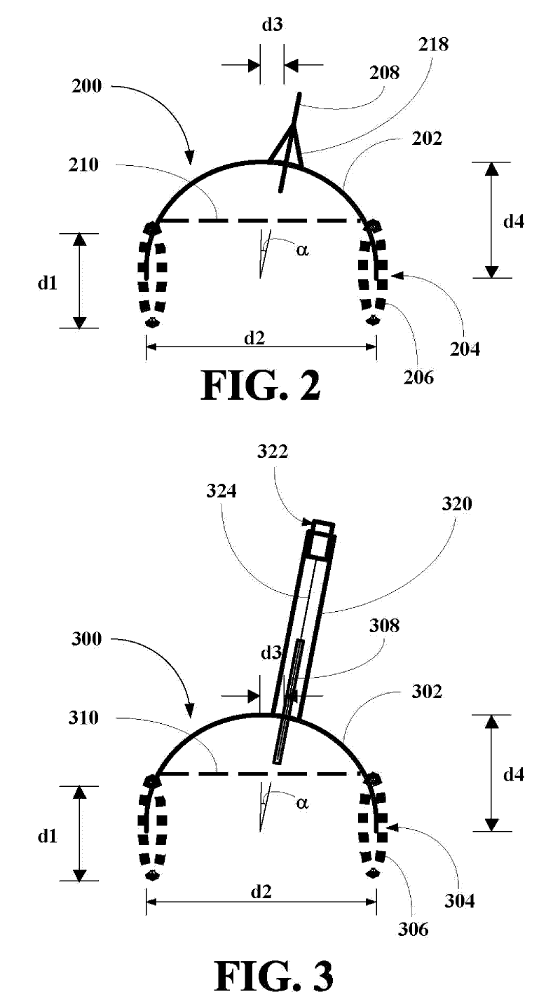

Referring now to FIG. 2, an embodiment of an intrathecal injection apparatus, generally 200, is shown to include a conforming member 202 adapted to conform to a portion of an animal back. The apparatus 200 also include two opposing apertures 204 surrounded by a flexible material member 206, where the two apertures 204 are shown here extending downward from the member 202. The apparatus 200 also include a needle guide tube 208. The apparatus 200 also include a fastening member or string 210. The apparatus 200 also includes a tube mount 218 adapted to mount the tube 208 to the member 202. The apparatus 200 includes all of the other components and parameters of the apparatus of FIGS. 1A-F.

Referring now to FIG. 3, an embodiment of an intrathecal injection apparatus, generally 300, is shown to include a conforming member 302 adapted to conform to a portion of an animal back. The apparatus 300 also include two opposing apertures 304 surrounded by a flexible material member 306, where the two apertures 304 are shown here extending downward from the member 302. The apparatus 300 also include a needle guide tube 308. The apparatus 300 also include a fastening member or string 310. The apparatus 300 also includes a needle and tube mount 320 adapted to mount the tube 308 to the member 302 and to house a needle 322 having a shaft 324 pre-inserted into the tube 308. The apparatus 300 includes all of the other components and parameters of the apparatus of FIGS. 1A-F. When the apparatus 300 is properly positioned on the animal and held in place by the string and the member 306, the needle 322 can be pushed down and penetrate into the spinal sack of the animal. The needle 322 can then be attached to an administration or withdrawal device.

All references cited herein are incorporated by reference. While this invention has been described fully and completely, it should be understood that, within the scope of the appended claims, the invention may be practiced otherwise than as specifically described. Although the invention has been disclosed with reference to its preferred embodiments, from reading this description those of skill in the art may appreciate changes and modification that may be made which do not depart from the scope and spirit of the invention as described above and claimed hereafter.

We claim:

1. An intrathecal injection guide apparatus comprising:
a body member adapted to conform to a portion of a lower torso of a small animal fitted with two opposing member adapted to engage opposing anatomical features on of the animal's lower torso to properly position the conforming member to the animal,
a guide member disposed on a top surface of the body member, and
a needle guide tube passing through the guide member and the body member, where the tube defines a proper needle trajectory or alignment so that when the apparatus is fitted onto the animal, a needle can simply be pushed to penetrate a spinal fluid sack of the animal at its distal end, where the sack lacks major neuron bundles.

2. The apparatus of claim 1, wherein the needle guide and the conforming member are a unitary assembly.

3. The apparatus of claim 1, wherein the needle guide is fixedly attached to the top surface of the conforming member.

4. The apparatus of claim 1, further comprising:
an empty or filled reservoir attached to a proximal end of the needle and adapted to permit a spinal material to be removed from the sack or a treating material to be injected into the sack.

5. An intrathecal injection guide apparatus comprising:
a body member adapted to conform to a portion of a lower torso of a small animal fitted with two opposing member adapted to engage opposing anatomical features on of the animal's lower torso to properly position the conforming member to the animal,
a guide member disposed on a top surface of the body member, and
a tube passing through the guide member and the body member including:
a needle housing having:
a needle pre-positioned in the housing with its tip positioned in a distal end of the tube,
where the tube defines a proper needle trajectory or alignment so that when the apparatus is fitted onto the animal, the needle can simply be pushed to penetrate a spinal fluid sack of the animal at its distal end, where the sack lacks major neuron bundles.

6. The apparatus of claim 5, wherein the needle guide and the conforming member are a unitary assembly.

7. The apparatus of claim 5, wherein the needle guide is fixedly attached to the top surface of the conforming member.

8. The apparatus of claim 5, further comprising:
an empty or filled reservoir attached to a proximal end of the needle and adapted to permit a spinal material to be removed from the sack or a treating material to be injected into the sack.

9. A method of transferring fluids in and out of the animal's spinal fluid comprising the step of:
fitting on a small animal an apparatus comprising:
a body member adapted to conform to a portion of a lower torso of a small animal fitted with two opposing member adapted to engage opposing anatomical features on of the animal's lower torso to properly position the conforming member to the animal,
a guide member disposed on a top surface of the body member, and
a needle guide tube passing through the guide member and the body member, where the tube defines a proper needle trajectory or alignment,
inserting a needle into the guide tube of the guide member, and
forcing the needle into a spinal sack of the animal in a distal location of the sack.

10. The method of claim 9, further comprising the step of:
attaching a proximal end of the needle to an empty or filled reservoir, and
either injecting a treating material into the sack or removing a spinal material from the sack.

11. The method of claim 9, further comprising the step of:
attaching a proximal end of the needle to an empty reservoir, and
removing a spinal material from the sack.

12. The method of claim 9, further comprising the step of:
attaching a proximal end of the needle to a filled reservoir, and
injecting a treating material into the sack.

13. The method of claim 9, wherein the guide member includes:
a needle housing having a needle pre-positioned in the housing with its tip positioned in a distal end of the tube.
